(19) United States
(12) Patent Application Publication
(10) Pub. No.: US 2010/0087727 A1
(43) Pub. Date: Apr. 8, 2010

(54) MULTI-MODALITY IMAGING APPARATUS AND METHOD FOR REGISTRATION OF THE MULTIPLE MODALITIES

(76) Inventor: Patrick Gross, Langensendelbach (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/561,582

(22) Filed: Sep. 17, 2009

(30) Foreign Application Priority Data
Sep. 17, 2008 (DE) 10 2008 047 644.7

(51) Int. Cl. A61B 5/055; G06K 9/00; A61B 8/00; A61B 6/00; A61N 7/00

(52) U.S. Cl. 600/411; 382/131; 600/439; 600/476; 601/2

(57) ABSTRACT

Method to register multiple different imaging modalities warming an examination subject in a common image acquisition region of all imaging modalities, simultaneous or time-offset acquisition of at least one first image data set with a first imaging modality, simultaneous or time-offset acquisition of at least one second heat image data set with a second imaging modality, determining at least one item of heat information resulting from the warming of the examination subject is determined from each of the first image data set and the second heat image data set, and registration of the imaging modalities using the determined heat information.

### BACKGROUND OF THE INVENTION

1. Field of the Invention

The invention concerns a method for registration two different imaging modalities in a combined system.

2. Description of the Prior Art

Various imaging methods are known in medical technology, for example computer tomography, magnetic resonance tomography and positron emission tomography. These imaging modalities deliver spatially resolved information about different physical properties of the examined region in the examination subject. In order to shorten measurement time and to achieve registration of two different modalities it is desirable to fashion these as a combined (also called a hybrid) modality.

Since the resolution and the position of the respectively acquired image data sets of a hybrid imaging modality normally do not coincide, and in addition not all imaging modalities can even be combined into hybrid units, a need exists for methods for registration of two different imaging modalities in order to be able to fuse their respective image data sets with each other.

Known methods for this purpose make use of anatomical landmarks (for example) that are imaged in the image data sets of the respective imaging modalities. For example, bones or even catheters can be used as landmarks. Bones, however, are stationary and catheters can only be invasively positioned, and in any case are dependent on the anatomy of the patient.

### SUMMARY OF THE INVENTION

An object of the present invention is to provide a method to register two different imaging modalities that allows the non-invasive use of landmarks at arbitrary points in the examination subject.

This object is achieved in accordance with the invention by a method that includes warming an examination subject in a common image acquisition region of both imaging modalities, simultaneous or time-offset acquisition of at least one image data set with the first imaging modality, simultaneous or time-offset acquisition of at least one heat image data set with the second imaging modality, determining at least one item of heat information resulting from the warming of the examination subject from each of the first image data set and the second heat image data set, registration of the imaging modalities using the determined heat information.

According to the invention it is thus provided to heat the examination subject in a targeted manner in order to place a landmark (a “heat landmark”, so to speak) within the body. Depending on the heating method used, this can be positioned arbitrarily and also reversibly since the heating of the tissue is dissipated again by diffusive compensation processes. Nevertheless, the landmark obtained in such a manner can be reproduced arbitrarily often, with care naturally being taken that the heated tissue is not damaged. Permissible durations and accumulations are well known from radiation and ultrasound therapy, so the present method can be implemented without risk to the patient.

Data sets from a region of the examination subject that contains the landmark can subsequently be acquired by arbitrarily many imaging modalities. In order to be able to conduct a registration using the thermal landmark, each of these data sets must contain heat information. For this purpose a data set can be a heat image data set. A “heat image data set” as used herein means a data set containing at least one item of heat information embodied in the image elements (pixels or voxels). The term does not mean that the heat information must necessarily be determined solely using a single data set. For example, a method for heat quantification is typical in magnetic resonance tomography, wherein phase differences between two image data sets acquired at different times are identified in order in order wherein a temperature change as heat information using these phase differences. One data set is thus required as a type of baseline while a second data set can be used for heat difference determination via a difference calculation. In this case, the base data set necessary for heat determination is naturally acquired before heating the examination subject, and so the heat image data set is the second data set acquired with the same modality. However, in this sense the reference data set without heating contains no heat information, and is acquired only once, and is therefore not considered herein as being encompassed by the heat image data set.

Neither a real heat imaging or heat change imaging is necessary, however, in order to determine the heat information. Heat information can already be derived from a single image data set, for example when the heating of the examination subject correlates with the signal intensity in the image data set. Qualitative information that can be evaluated is still present in the image data set, even if a quantification does not exist.

At least one item of heat comparison information can advantageously be determined from the heat information of the first image data set (composed of image elements) and second heat image data set. This means that a specific property of the heat information or heat distribution is extracted from the image data for registration.

A simpler case results when both imaging modalities are capable of heat imaging. An imaging modality capable of heat imaging can then also be used as a first imaging modality to acquire a first heat image data set, so each of the first and second heat image data sets contains direct heat or heat change information. The determination and the comparison of heat information is thereby particularly simple.

The warmest image element can advantageously be used as heat comparison information in the heat image data sets. The warmest image element identifies either the highest temperature or the greatest temperature change. Due to the heating of the examination subject, it is assumed that the warmest position in the image data set is located at the point of the heating, and not within a region that is warm anyway. The warmest image element is correspondingly suitable to represent the center of the heat, and the registration is to be achieved using this landmark. However, it should be noted that a single point alone is not yet sufficient for registration. Assuming that the heat image data sets map the same plane, two points or a point and a direction specification connected with it are sufficient in order to achieve a complete registration. The warmest image element thus serves as one item of heat comparison information.

Due to noise, however, it can be appropriate to interpolate the heat distribution present in the heat image data set (the distribution function of which is typically known) and to determine the warmest image element using the interpolation.

Since, as described above, a single point is not sufficient for registration, one or more temperature gradients can be respectively determined as possible additional heat comparison information in the heat image data sets as an alternative or in addition to the warmest image element. A gradient results by taking the difference between at least two points; but it can also be interpolated over multiple points. The registration in this case can therefore already ensue using a single temperature gradient, but for compensation of measurement inaccuracies it is advantageous to determine multiple temperature gradients and to have a registration occur using an interpolation.

In addition to the temperature gradient as a possible additional item of heat comparison information in the heat image data sets, at least one anatomical landmark that is likewise imaged in the respective heat image data set can also be particularly advantageously used. The use of anatomical landmarks is generally known to those skilled in the art, but in some cases (for example given rotationally symmetrical landmarks) an unambiguous registration is not possibly solely using this anatomical landmark. In this case, the landmark generated based on the heating of the examination subject can be used in addition, with the conventional anatomical landmark being a bone, for example, so with the landmark based on the heating, at least two points are thus available for registration.

As already described, a certain interpolation or averaging is suitable to improve the comparison result. The focal point of the heat distribution in at least one sub-region of the heat image data set can be particularly advantageously determined as additional heat comparison information in the heat image data set. This focal point forms only one of the two necessary points for registration, but it is weighted across all or at least a portion of the heat information, which is why it can be determined with low errors. In particular, the focal point can be less affected by the image resolution or the slice thickness, which is why it is particularly suitable for comparison of heat image data sets with different resolution properties.

A device that is based on the same physical effect as one of the two imaging modalities can advantageously be used to heat the examination subject in the image acquisition region. This means that, for example, the heating is implemented by a therapeutic ultrasound apparatus, and one of the two imaging modalities is then an imaging ultrasound device. Another possible configuration is the use of a light source (in particular a laser) with an optical acquisition device (for example a camera) being used for imaging.

In this case, in an alternative embodiment for acquisition of a first and second heat image data set it is possible to acquire only one or multiple first image data sets with the first imaging modality, these first image data sets allowing no direct quantification of heat information. In this case, the heat information of the first image data set can be determined using the distribution of the signal intensity in the image elements of the first image data set.

In this case the aforementioned possibilities to determine heat comparison information from the first image data set differ from the determination of heat comparison information from the second heat image data set to the extent that the heat comparison information is determined from the signal intensity instead of from a numerical temperature value, or using one or more signal intensity gradients instead of temperature gradients. This is based on the assumption that the signal intensity and the temperature, or the changes in difference data sets, are proportional to one another at least in a portion of the image, and that this information is already sufficient for registration even without knowledge of the proportionality factor that would be necessary for quantification.

To calculate the focal point and to determine the additional aforementioned heat comparison information, the question naturally exists as to which pixels are to be included in the analysis and which are not. Therefore, at least one threshold can be used to determine the heat information, and only heat information that is above or below this threshold is used. For example, by the use of the threshold, only heated regions of the examination subject are included in the analysis. Also by the use of the threshold, only regions whose temperature increase exhibits a certain minimum deviation from the average temperature can be included. Furthermore, a maximum value can be established, so miscalculations in the form of temperature spikes due to measurement inaccuracies are likewise precluded.

As described above, landmarks can be placed in the examination subject at an arbitrary location and at arbitrary points in time by the heating. The heating of the examination subject can be varied between the acquisition of at least two image data sets or heat image data sets of an imaging modality. Multiple advantages can be realized by this procedure. Furthermore, the possibility to register the imaging modalities is provided through the variation of the point of the heating of the examination subject, and a type of time stamp can therefore simultaneously be imprinted on the image. Namely, once first and second image or heat image data sets with an identical landmark due to the heating exist, the registration of the imaging modalities is already provided. The heat information of the additional heat image data sets can correspondingly be used to establish the point in time of the acquisition.

The method is naturally not limited to the two-dimensional image data sets. For three-dimensional registration, the establishment of three points is required, which can ensue due to the spatial distribution of a one-time heating of the examination subject and with consideration of possible additional anatomical landmarks imaged in the heat image data set. It is also possible to heat the examination subject at three points for this purpose. A three-dimensional registration is also possible by suitable shaping of the heated region.

In another embodiment, at least one additional image data set or heat image data set is acquired after the registration by using one of the imaging modalities, and by comparison with the first or last acquired image data set or heat image data set of this imaging modality, movement information is determined with which the image data acquisition or the image data evaluation of the other imaging modality is movement-corrected. For example, this procedure makes sense if the one of the two imaging modalities has a much higher time resolution and the second imaging modality requires either multiple passes to acquire an image data set, or multiple image data sets for averaging. If the analysis of the heat image data sets of the one imaging modality ensues automatically and without a time loss, the data acquisition of the other imaging modality can already ensue with movement correction. A movement correction only in the form of post-processing steps is also possible.

The invention also encompasses a combined imaging device to implement the method, this combined imaging device containing the two imaging modalities as well as a heat generation device, so heat image data sets can be acquired with at least one of the two imaging modalities. The heat generation device and one of the two imaging modalities can advantageously be based on the same physical effect. Ultrasound was already cited above as an example. This embodiment allows the implementation of particularly compact combined devices for simultaneous heat generation and heat-dependent imaging.

Alternatively, the heat generation device can be a light source and the imaging modality can be an optical imaging device. For example, it is possible to heat an object with a laser beam within the framework of materials testing and to capture this procedure with a camera. The image data of the camera do not allow temperature determination but display the light intensity distribution of the laser light very well. Additionally acquired heat image data sets (for example of a magnetic resonance system) can be registered with the image data sets of the camera with the method according to the invention.

An imaging modality can particularly advantageously be a magnetic resonance device. Multiple methods for temperature determination are known from magnetic resonance tomography. For example, it is known to acquire data sets with heat information via T₁ maps or phase difference imaging, or even spectroscopic imaging.

### BRIEF DESCRIPTION OF THE DRAWINGS

FIG. 1 illustrates a combined imaging device.

FIG. 2 shows an embodiment of a workflow for operation of the combined imaging device.

FIG. 3 illustrates the registration of the imaging modalities.

### DESCRIPTION OF THE PREFERRED EMBODIMENTS

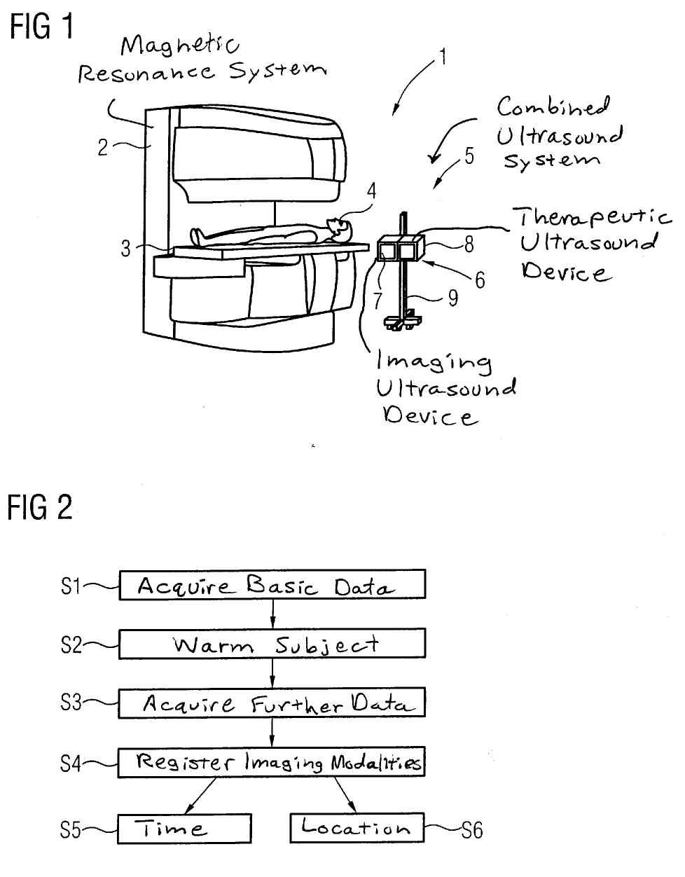

The combined imaging device 1 according to FIG. 1 has a magnetic resonance system 2 with a patient bed 3 on which the patient 4 is borne, as well as a combined ultrasound system 5. In the combined ultrasound system 5, both an imaging ultrasound device 7 and a therapeutic ultrasound device 8 for heating tissue are arranged in a housing 6. Furthermore, a support 9 is provided for positioning at the patient. Details with regard to the magnetic resonance system 2 and the combined ultrasound device 5 that are well known to those skilled in the art and need not be specifically shown. Among these are, for example, a common control device or multiple control devices, at least one radio-frequency coil of the magnetic resonance system 2, etc. In one embodiment, the combined ultrasound device 5 can also be attached to a robot arm that itself can in turn be attached to the housing 6 of the magnetic resonance system. The combined ultrasound system 5 can be positioned both by a user as well as automatically via such a robot arm. For example, it is possible to first acquire an overview scan with the magnetic resonance system 2 in order to establish the examination center (automatically or by a user) and then to automatically position both the patient bed 3 and the combined ultrasound system 5. Although a certain registration is already provided by this procedure, the method according to the invention allows registration with precision to the image element that, without the method according to the invention, could only be achieved with high-precision sensor devices to determine the position of the devices.

The workflow scheme FIG. 2 for operation of the combined imaging device 1 includes the acquisition of basic data as a first Step S1. This means that a basic or reference data set is acquired, for example for temperature determination with a magnetic resonance system 2. An additional data set is acquired after acquisition of the reference data set and after a variation of the temperature distribution in the measured region of the examination subject in Step 2. These can be simple image data sets with phase information or even complete T₁ maps. By comparison of the second data sets with the reference data sets, a temperature change can then be established by taking the difference. Without heating of the examination subject, apart from noise the data sets should contain the same data and be more or less identical. In order to detect the time progression of temperature changes, only a repetition of the acquisition of the respective data set is necessary. A temperature change determination can be done by comparison with the reference data set or even with the last or any other previously acquired data set. Naturally, these are respective data sets with the same information, for example thus Tᵢ maps. In the case of a temperature determination via a difference method, this Step (executed under S3) thus describes the acquisition of an additional image data set via the respective imaging modality in order to then obtain the heat image data set by taking the difference with the reference image data set.

In the event that it is possible to already obtain a heat image data set from a single image data set, Step S1 can be omitted. In every instance, after Step S3 one heat image data set per imaging modality is available. These are then used in Step S4 to register the imaging modalities with one another. After registration has occurred, there are multiple possibilities to proceed further. In a first embodiment, additional heat image data sets can be acquired with one or more imaging modalities, wherein the heating location in the examination subject is varied between the acquisitions. A heating generated once in the examination subject is naturally lost again due to heat exchange processes within the examination subject. It is therefore possible to heat the examination subject at locations that are new every time, and to then use this time progression to temporally register the heat image data sets. This time registration is shown as Step S5.

In an alternative embodiment, in S6 the location of the heating is kept constant while possible location changes in the heat image data sets can then be ascribed to a movement of the examination subject. Temporally high-resolution heat image data sets (that were acquired by means of an imaging ultrasound, for example) can be used to subject the data set exposure of an image data set of the magnetic resonance system 2 to a movement correction. This data set can be an arbitrary data set; it does not have to be a heat image data set and also does not have to be an image data set; for example, it can be a spectroscopic data set. A movement correction is thereby necessary for spatially selective spectroscopy, for example.

Step S1 can also be omitted if, although the image data set acquired under S3 does not allow heat quantification, the signal intensity for this is proportional to the heating in at least one region of the image data set. For example, this is the case in the aforementioned situation of a laser and a camera, even if a therapeutic ultrasound whose reflection signals are detected by an imaging ultrasound is used for heating. A greater heating of the tissue is achieved at the points of the tissue at which the sound waves introduced by the therapeutic ultrasound achieve a higher density or intensity, while a greater reflection signal is to be expected in the imaging ultrasound. The signal intensity and the heating of the tissue are thus correlated.

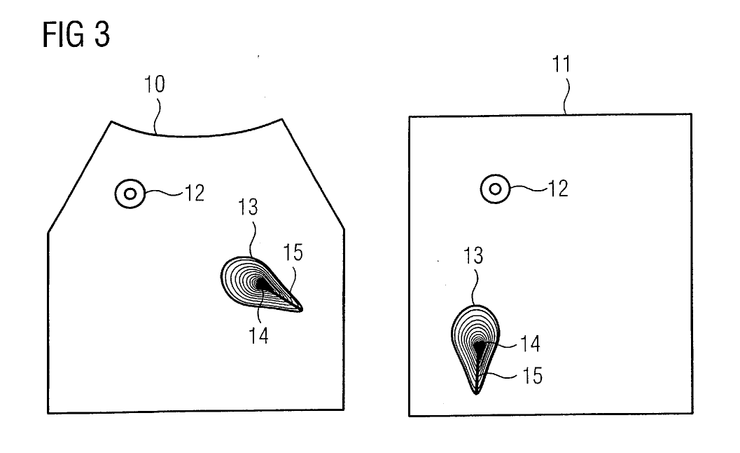

One possibility to register the imaging modalities of the combined imaging device 1 is shown in FIG. 3. The heat image data set 10 acquired with the imaging ultrasound device 7 and the heat image data set 11 acquired with the magnetic resonance system 2 respectively show a bone 12 and a heat landmark 13. This heat landmark is created in that the examination subject has been specifically heated with a therapeutic ultrasound in a region that is imaged in the heat image data sets 10 and 11. The registration then proceeds as follows. A threshold is first established that specifies which values are taken into account in the temperature change. In the heat image data sets 10 and 11 only the heat landmark 13 is accordingly visible. The bone shows no temperature change at all and is accordingly no longer imaged after setting the threshold. A defined point is then established starting from the heat landmark 13. For example, the image element 14 is characterized as exhibiting the greatest temperature change. The image elements determined from the heat image data sets can be used, but the heat distribution function of the heat landmarks 13 is also known, so the heat landmark 13 can be subjected to an interpolation in order to determine possible measurement inaccuracies from this. Starting from the image element 14, the direction of the furthest expansion of the heat landmark 13 can be determined in every case. This is represented by the line 15. A simple registration of the imaging modalities is thereby possible via the image element 14 and the line 15.

As an alternative to this procedure, in addition to the image element 14 the bone 12 can also be used for registration, and instead of the image element with the greatest temperature change the focal point of the heat distribution could also be calculated from the heat landmark 13. The more image elements that are used for an interpolation or averaging, the smaller the inaccuracies as a result of measurement errors. As a minimum, two points or one point and a direction specification are required for a registration of two-dimensional image data sets, and three points or one point with two direction vectors or two pixels and one direction specification belonging to one point are required for a registration with the aid of three-dimensional image data sets.

The embodiments of the imaging modalities capable of heat imaging as a magnetic resonance system 2 and ultrasound system 5 are only exemplary; a light source could also be combined with an optical imaging device instead of the combined ultrasound system 5.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method to bring two different imaging modalities into registration with each other, comprising the steps of:
warming an examination subject in a common image acquisition region of a first imaging modality and a second imaging modality;
acquiring an image data set of the examination subject in the image acquisition region;
before, after or simultaneously with obtaining said image data set with said first imaging modality, acquiring a heat image data set of the examination subject in the image acquisition region with said second imaging modality;
determining at least one item of heat information, resulting from said warming of the examination subject, from each of said image data set and said heat image data set; and
bringing the first and second imaging modalities into registration with each other using said heat information respectively determined from said image data set and said heat image data set.

2. A method as claimed in claim 1 wherein each of said image data set and said heat image data set is comprised of image elements, selected from the group consisting of pixels and voxels, and comprising determining said item of heat information from each of said image data set and said heat image data set as an image element-dependent item of heat information.

3. A method as claimed in claim 2 comprising employing an imaging modality operable for heat imaging as said first imaging modality, and acquiring said image data set with said first imaging modality also as a heat image data set.

4. A method as claimed in claim 3 comprising identifying a warmest image element in each of the heat image data sets respectively acquired with said first and second imaging modality, and bringing said first and second imaging modalities into registration by comparing the respective warmest image elements in the respective heat image data sets.

5. A method as claimed in claim 3 comprising identifying a temperature gradient in each of the heat image data sets respectively acquired with said first and second imaging modality, and bringing said first and second imaging modalities into registration by comparing the respective temperature gradients in the respective heat image data sets.

6. A method as claimed in claim 5 comprising, with each of said first and second imaging modalities, imaging an anatomical landmark in the examination subject in the respective heat image data sets, and bringing said imagining modalities into registration using said anatomical landmark in the respective heat image data sets, in addition to said heat information.

7. A method as claimed in claim 1 comprising identifying, as said item of heat information in each of said image data set and said heat image data set, a focal point of heat distribution in at least one region of each of said image data set and said heat image data set, and bringing said first and second imaging modalities into registration by comparing the respective focal points of heat distribution in the image data set and the heat image data set.

8. A method as claimed in claim 1 wherein said first imaging modality acquires said image data set by a first physical interaction with the examination subject and wherein said imaging modality acquires said heat image data set by a second physical interaction with the examination subject, and comprising warming the examination subject using a warming modality that employs either said first physical interaction or said second physical interaction to warm the examination subject.

9. A method as claimed in claim 8 wherein said image data set if comprised of image elements selected from the group consisting of pixels and voxels, and comprising warming said examination subject with an imaging modality that operates with an image element-dependent distribution of signal intensity applied to the examination subject, and comprising determining said item of heat information from said image data set using said distribution of the signal intensity.

10. A method as claimed in claim 9 comprising identifying an image element having a highest signal intensity associated therewith in said image data set, and identifying a warmest image element in said heat image data set, and bringing first and second modalities into registration by comparing said image element having the highest signal intensity with the warmest image element.

11. A method as claimed in claim 9 comprising identifying at least one signal intensity gradient in said image data set and at least one temperature gradient in said heat image data set, and bringing said first and second modalities into registration by comparing said at least one signal intensity gradient and said at least one temperature gradient.

12. A method as claimed in claim 11 comprising acquiring an image of an anatomical landmark in the examination subject in each of said image data set and said heat image data set, and brining said first and second modalities into registration using said anatomical landmark in addition to said comparison of said at least one signal intensity gradient and said at least one temperature gradient.

13. A method as claimed in claim 9 comprising identifying a focal point of said signal intensity distribution in said image data set and identifying a focal point of heat distribution in said heat image data set, and bringing said first and second modalities into registration by comparing said focal point of said signal intensity distribution and said focal point of heat distribution.

14. A method as claimed in claim 9 wherein said heat image data set has a heat distribution therein, and comprising determining said item of heat information from said image data set using only a portion of said signal intensity distribution that is above a predetermined signal intensity threshold, and determining said item of heat information from said heat image data set using only a portion of said heat distribution that is above a predetermined heat threshold.

15. A method as claimed in claim 9 wherein said heat image data set has a heat distribution therein, and comprising determining said item of heat information from said image data set using only a portion of said signal intensity distribution that is below a predetermined signal intensity threshold, and determining said item of heat information from said heat image data set using only a portion of said heat distribution that is below a predetermined heat threshold.

16. A method as claimed in claim 1 comprising non-uniformly warming the examination subject between a time of acquisition of said image data set and a time of acquisition of said heat image data set.

17. A combined imaging device comprising:
a first imaging modality and a second imaging modality that sharer a common image acquisition region;
a heat generator that interacts with an examination subject in said image acquisition region to warm the examination subject in said image acquisition region;
a control unit that operates said first imaging modality and said second imaging modality to acquire an image data set with the first imaging modality and to acquire a heat image data set with the second imaging modality, said image data set and said heat image data set being acquired either simultaneously or in succession; and
a processor supplied with said image data set and said heat image data set that determines an item of heat information from said image data set and an item of heat information from said heat image data set resulting from said warming of said examination subject, and that brings said first and second imaging modalities into registration using said heat information from said image data set and said heat information from said heat image data set.

18. A combined imaging device as claimed in claim 17 wherein said heat generator is a therapeutic ultrasound device and wherein at least one of said first and second imaging modalities is an ultrasound imaging device.

19. A combined imaging device as claimed in claim 17 wherein said heat generator is a light source, and wherein at least one of said first and second imaging modalities is an optical imaging device.

20. A combined imaging device as claimed in claim 17 wherein one of said first and second imaging modalities is a magnetic resonance imaging device.
